(19) United States
(12) Patent Application Publication
(10) Pub. No.: US 2021/0122777 A1
(43) Pub. Date: Apr. 29, 2021

(54) A PROCESS FOR PREPARING KETOLIDE COMPOUNDS

(71) Applicant: WOCKHARDT LIMITED, Chikalthana, Aurangabad (IN)

(72) Inventors: Furqan Mohammed DIWAN, Buldhana 5 (IN); Amit PUND, Nashik 2 (IN); Mohammad RAFEEQ, PIlbhit 1 (IN); Arvind Yekanathsa MERWADE, Belgaum 2 (IN)

(73) Assignee: WOCKHARDT LIMITED, Chikalthana, Aurangabad (IN)

(21) Appl. No.: 16/492,145

(22) PCT Filed: Mar. 14, 2018
(86) PCT No.: PCT/IB2018/051682

§ 371 Date: Sep. 8, 2019

(30) Foreign Application Priority Data
Mar. 16, 2017 (IN) 201721009182

(51) Int. Cl. C07H 17/08

(52) U.S. Cl. CPC C07H 17/08

(57) ABSTRACT

A process for preparing ketolide compounds is disclosed.

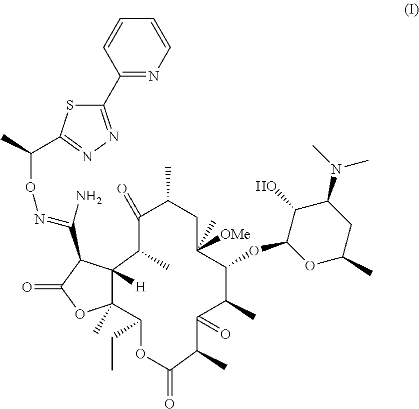 [SMILES: CC[C@@H]1OC(=O)[C@H](C)C(=O)[C@H](C)[C@@H](O[C@@H]2O[C@H](C)C[C@H](N(C)C)[C@H]2O)[C@](C)(OC)C[C@@H](C)C(=O)[C@H](C)[C@H]2[C@H](/C(N)=N/O[C@@H](C)c3nnc(-c4ccccn4)s3)C(=O)O[C@@]21C]

### RELATED PATENT APPLICATIONS

This application claims priority to and benefit of the Indian Patent Application No. 201721009182 (filed Mar. 16, 2017), the disclosures of which is incorporated herein by reference in its entirety as if fully rewritten herein.

### FIELD OF THE INVENTION

The invention relates to a process for preparing ketolide compounds.

### BACKGROUND OF THE INVENTION

PCT International Patent Applications PCT/M2010/052325 and PCT/M2011/050464 disclose several ketolide compounds having antibacterial properties. The present invention discloses an improved process for preparing such and other ketolide compounds.

### SUMMARY OF THE INVENTION

Accordingly, there is provided a process for preparing ketolide compounds.

In one general aspect, there is provided a process for preparing a compound of Formula (I):

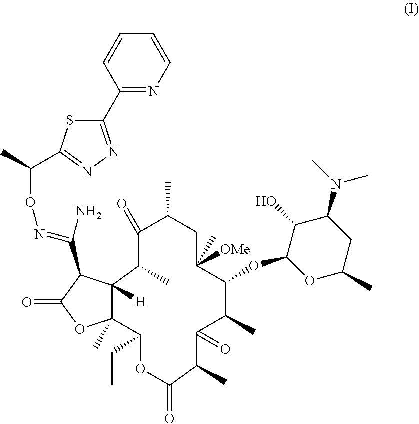 [SMILES: CC[C@@H]1OC(=O)[C@H](C)C(=O)[C@H](C)[C@@H](O[C@@H]2O[C@H](C)C[C@H](N(C)C)[C@H]2O)[C@](C)(OC)C[C@@H](C)C(=O)[C@H](C)[C@H]2[C@H](/C(N)=N/O[C@@H](C)c3nnc(-c4ccccn4)s3)C(=O)O[C@@]21C]

In another general aspect, there is provided a process for preparing a compound of Formula (VII):

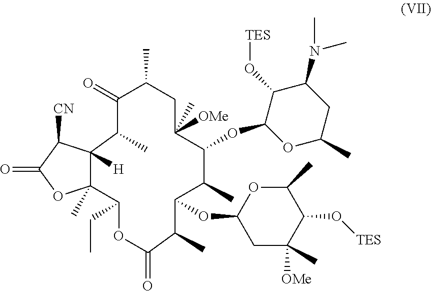

The details of one or more embodiments of the invention are set forth in the description below. Other features, objects and advantages of the invention will be apparent from the following description, including claims.

### DETAILED DESCRIPTION OF THE INVENTION

Reference will now be made to the exemplary embodiments, and specific language will be used herein to describe the same. It should nevertheless be understood that no limitation of the scope of the invention is thereby intended. Alterations and further modifications of the inventive features illustrated herein, and additional applications of the principles of the invention as illustrated herein, which would occur to one of ordinary skills in the relevant art and having possession of this disclosure, are to be considered within the scope of the invention. It must be noted that, as used in this specification and the appended claims, the singular forms “a,” “an,” and “the” include plural referents unless the content clearly dictates otherwise. All references including patents, patent applications, and literature cited in the specification are expressly incorporated herein by reference in their entirety.

In one general aspect, there is provided a process for preparing a compound of Formula (I),

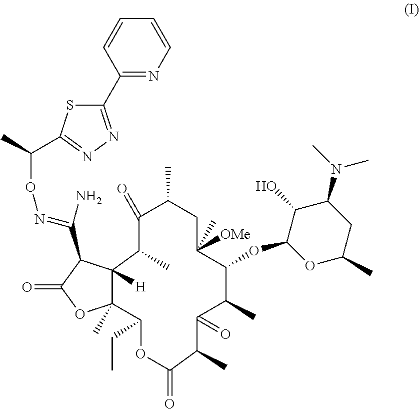 [SMILES: CC[C@@H]1OC(=O)[C@H](C)C(=O)[C@H](C)[C@@H](O[C@@H]2O[C@H](C)C[C@H](N(C)C)[C@H]2O)[C@](C)(OC)C[C@@H](C)C(=O)[C@H](C)[C@H]2[C@H](/C(N)=N/O[C@@H](C)c3nnc(-c4ccccn4)s3)C(=O)O[C@@]21C]

said process comprising:

(a) obtaining a compound of Formula (III) by reacting a compound of Formula (II) with triethylsilyl chloride in presence of a base and a solvent;

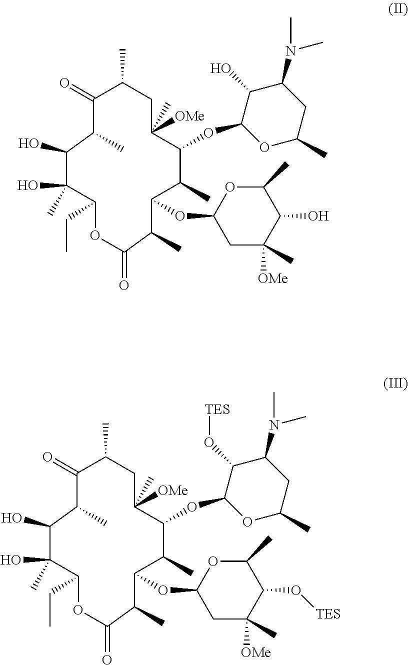

(b) obtaining a compound of Formula (IV) by reacting the compound of Formula (III) with triphosgene in presence of a base and a solvent;

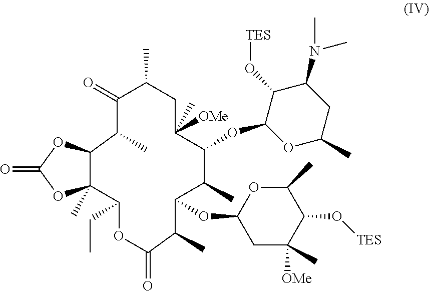

(c) obtaining a compound of Formula (V) by reacting the compound of Formula (IV) with a base in presence of a solvent;

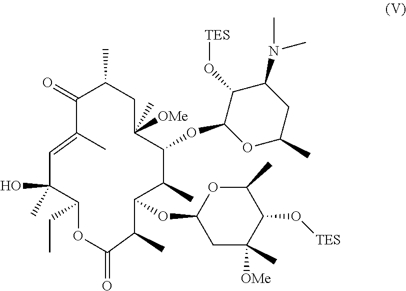

(d) obtaining a compound of Formula (VI) by reacting the compound of Formula (V) with chloroacetic acid and 4-dimethylaminopyridine in presence of a coupling agent and a solvent;

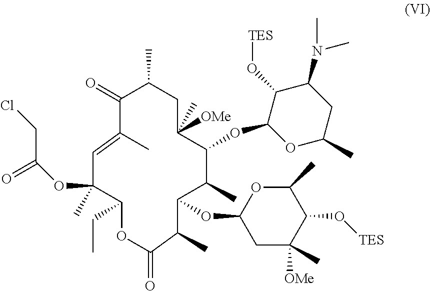

(e) obtaining a compound of Formula (VII) by reacting the compound of Formula (VI) with trimethylsilyl cyanide in presence of a base, an activating agent and a solvent;

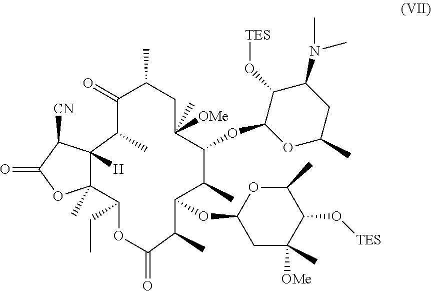

(f) obtaining a compound of Formula (VIII) by treating the compound of Formula (VII) with hydrochloric acid in presence of a solvent; followed by treatment with triethysilyl chloride in presence of a base and a solvent;

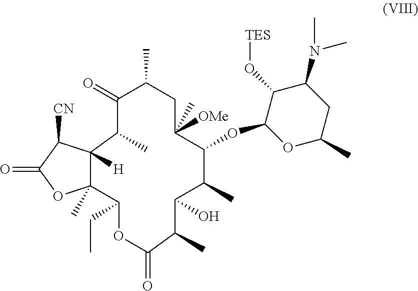

(g) obtaining a compound of Formula (IX) by treating the compound of Formula (VIII) with hydroxylamine hydrochloride in presence of a base and a solvent;

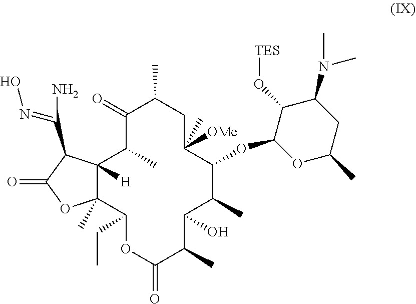

(h) obtaining a compound of Formula (XI) by treating the compound of Formula (IX) with a compound of Formula (X) in presence of 18-crown-6 ether, a base and a solvent;

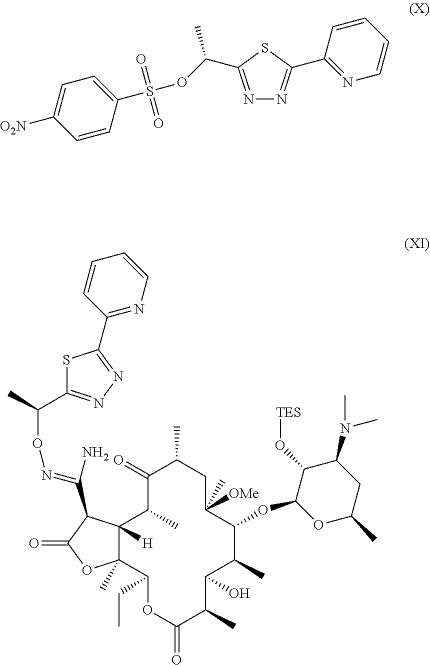

(i) obtaining a compound of Formula (XII) by reacting the compound of Formula (XI) with N-chlorosuccinimide in presence of dimethyl sulfide, a base and a solvent; and

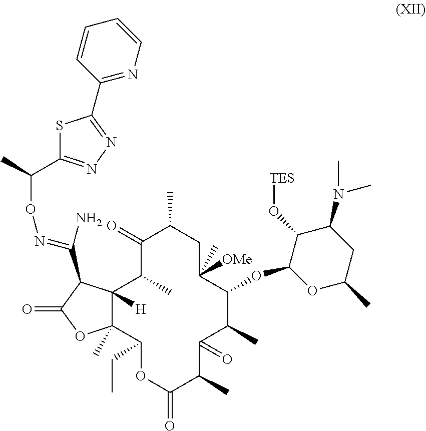

(j) obtaining the compound of Formula (I) by de-protecting the compound of Formula (XII).

In another general aspect, there is provided a process for preparing a compound of Formula (VII), said process comprising reacting a compound of Formula (VI) with trimethylsilyl cyanide in presence of a base, an activating agent and a solvent.

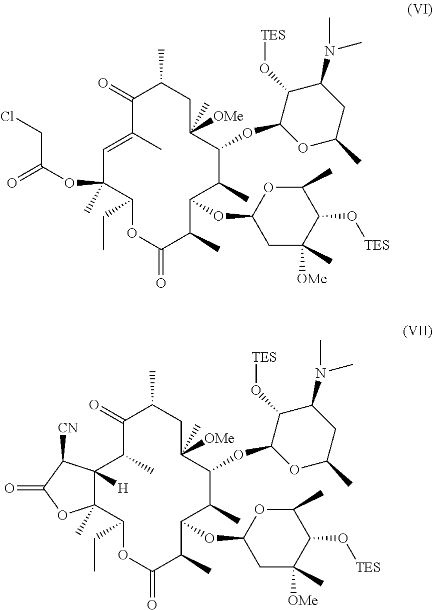

Step (a): Preparation of a Compound of Formula (III)

In general, the compound of Formula (III) is obtained by reacting the compound of Formula (II) with triethylsilyl chloride in presence of a base and a solvent:

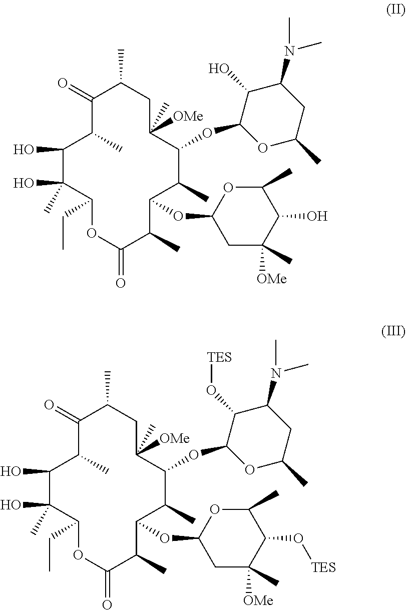

A wide variety of bases and solvents can be used in this step. Typical, non-limiting examples of bases that can be used in this step include triethylamine, 4-dimethylaminopyridine, N,N-diisopropylethylamine, or a mixture thereof. Typical, non-limiting examples of solvents that can be used in this step include N,N-dimethylformamide, dichloromethane, acetonitrile, N-methylpyrrolidine, tetrahydrofuran, ethylacetate, acetone, dimethyl sulfoxide or a mixture thereof. The reaction may be carried out at a wide range of temperatures. In some embodiment, this reaction is carried out at a temperature between 5° C. to 30° C. In some other embodiments, this reaction is carried out at a temperature between 5° C. to 10° C.

Step (b): Preparation of a Compound of Formula (IV)

In general, the compound of Formula (IV) is obtained by reacting the compound of Formula (III) with triphosgene in presence of a base and a solvent:

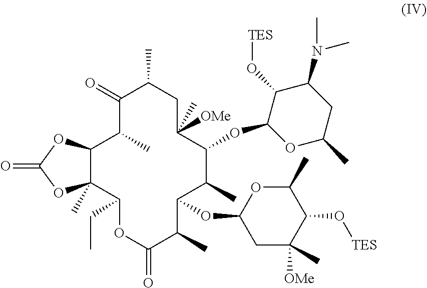

A wide variety of bases and solvents can be used in this step. Typical, non-limiting examples of bases that can be used in this step include pyridine, triethylamine, 4-dimethylaminopyridine, N,N-diisopropylethylamine, or a mixture thereof. Typical, non-limiting examples of solvents that can be used in this step include dichloromethane, N,N-dimethylformamide, acetonitrile, N-methylpyrrolidine, tetrahydrofuran, ethylacetate, acetone, dimethyl sulfoxide or a mixture thereof. The reaction may be carried out at a wide range of temperatures. In some embodiment, this reaction is carried out at a temperature between 5° C. to 30° C. In some other embodiments, this reaction is carried out at a temperature between 5° C. to 10° C.

Step (c): Preparation of a Compound of Formula (V)

In general, the compound of Formula (V) is obtained by reacting the compound of Formula (IV) with a base in presence of a solvent:

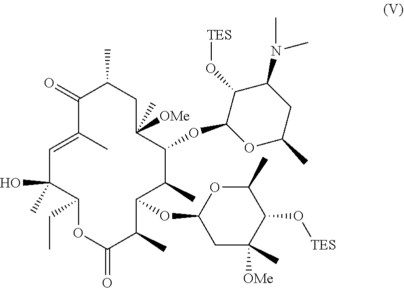

A wide variety of bases and solvents can be used in this step. Typical, non-limiting examples of bases that can be used in this step include 1,8-diazabicyclo[5.4.0]undec-7-ene, triethylamine, N,N-diisopropylethylamine, 1,5-diazabicyclo(4.3.0)non-5-ene, or a mixture thereof. Typical, non-limiting examples of solvents that can be used in this step include acetone, dichloromethane, N,N-dimethylformamide, acetonitrile, N-methylpyrrolidine, tetrahydrofuran, ethylacetate, dimethyl sulfoxide or a mixture thereof. The reaction may be carried out at a wide range of temperatures. In some embodiment, this reaction is carried out at a temperature between 5° C. to 30° C. In some other embodiments, this reaction is carried out at a temperature between 20° C. to 30° C.

Step (d): Preparation of a Compound of Formula (VI)

In general, the compound of Formula (VI) is obtained by reacting the compound of Formula (V) with chloroacetic acid and 4-dimethylaminopyridine in presence of a coupling agent and a solvent:

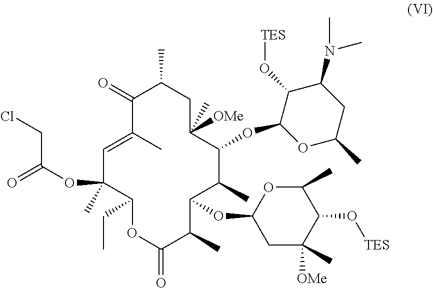

A wide variety of coupling agents and solvents can be used in this step. Typical, non-limiting examples of coupling agents that can be used in this step include N,N′-dicyclohexylcarbodiimide (DCC), N-(3-Dimethylaminopropyl)-N′-ethylcarbodiimide hydrochloride (EDCI), 1-Hydroxybenzotriazole (HOBT), N-[(Dimethylamino)-1H-1,2,3-triazolo-[4,5-b]pyridin-1-ylmethylene]-N-methylmethanaminium hexafluorophosphate N-oxide (HATU), N,N,N′,N′-Tetramethyl-O-(1H-benzotriazol-1-yl)uronium hexafluorophosphate (HBTU) or a mixture thereof. Typical, non-limiting examples of solvents that can be used in this step include dichloromethane, acetone, N,N-dimethylformamide, acetonitrile, N-methylpyrrolidine, tetrahydrofuran, ethylacetate, dimethyl sulfoxide or a mixture thereof. The reaction may be carried out at a wide range of temperatures. In some embodiment, this reaction is carried out at a temperature between 0° C. to 30° C. In some other embodiments, this reaction is carried out at a temperature between 0° C. to 10° C.

Step (e): Preparation of a Compound of Formula (VII)

In general, the compound of Formula (VII) is obtained by reacting the compound of Formula (VI) with trimethylsilyl cyanide in presence of a base, an activating agent and a solvent:

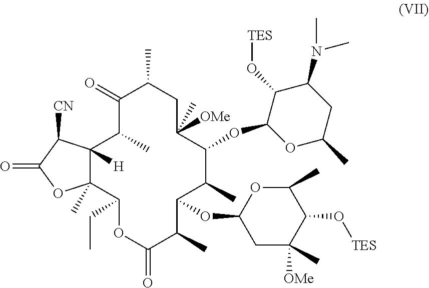

A wide variety of bases, activating agents and solvents can be used in this step. Typical, non-limiting examples of bases that can be used in this step include cesium carbonate, sodium carbonate, potassium carbonate, sodium hydroxide, potassium hydroxide, lithium hydroxide, sodium methoxide, potassium tert-butoxide, sodium ethoxide, or a mixture thereof. Typical, non-limiting examples of activating agents that can be used in this step include C₁-C₆ alcohol, water or a mixture thereof. In some embodiments, the activating agent used is methanol. Typical, non-limiting examples of solvents that can be used in this step include N,N-dimethylformamide, N-methylpyrrolidine, tetrahydrofuran, ethylacetate, acetone, acetonitrile, dimethyl sulfoxide, or a mixture thereof. In some embodiments, the solvent used is N,N-dimethylformamide. The reaction may be carried out at a wide range of temperatures. In some embodiment, this reaction is carried out at a temperature between 0° C. to 50° C. In some other embodiments, this reaction is carried out at a temperature between 40° C. to 50° C.

Step (f): Preparation of a Compound of Formula (VIII)

In general, the compound of Formula (VIII) is obtained by treating the compound of Formula (VII) with hydrochloric acid in presence of a solvent; followed by treatment with triethysilyl chloride in presence of a base and a solvent;

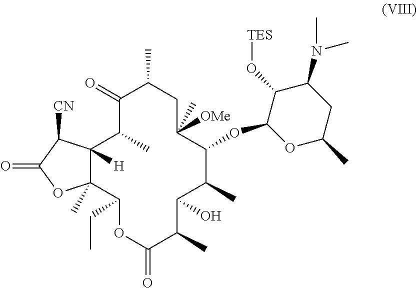

A wide variety of bases and solvents can be used in this step. Typical, non-limiting examples of solvents that can be used in this reaction include methanol, ethanol, isopropyl alcohol, N,N-dimethylformamide, dichloromethane, acetonitrile, N-methylpyrrolidine, tetrahydrofuran, ethylacetate, acetone, dimethyl sulfoxide, or a mixture thereof. Typical, non-limiting examples of bases that can be used in this step include triethyl amine, 4-dimethylaminopyridine, N,N-diisopropylethylamine, or a mixture thereof. The reaction may be carried out at a wide range of temperatures. In some embodiment, this reaction is carried out at a temperature between 0° C. to 50° C. In some other embodiments, this reaction is carried out at a temperature between 30° C. to 40° C.

Step (g): Preparation of a Compound of Formula (IX)

In general, the compound of Formula (IX) is obtained by treating the compound of Formula (VIII) with hydroxylamine hydrochloride in presence of a base and a solvent:

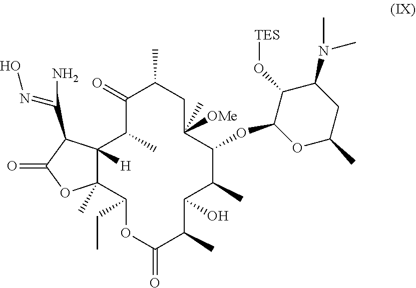

A wide variety of bases and solvents can be used in this step. Typical, non-limiting examples of bases that can be used in this step include sodium carbonate, potassium carbonate, cesium carbonate, sodium hydroxide, potassium hydroxide, lithium hydroxide, sodium methoxide, potassium tert-butoxide, sodium ethoxide, pyridine, triethylamine, 4-dimethylaminopyridine, N,N-diisopropylethylamine, or a mixture thereof. Typical, non-limiting examples of solvents that can be used in this step include methanol, ethanol, isopropyl alcohol, dichloromethane, acetone, N,N-dimethylformamide, acetonitrile, N-methylpyrrolidine, tetrahydrofuran, dimethyl sulfoxide, or a mixture thereof. The reaction may be carried out at a wide range of temperatures. In some embodiment, this reaction is carried out at a temperature between 0° C. to 50° C. In some other embodiments, this reaction is carried out at a temperature between 30° C. to 40° C.

Step (h): Preparation of a Compound of Formula (XI)

In general, the compound of Formula (XI) is obtained by treating the compound of Formula (IX) with a compound of Formula (X) in presence of 18-crown-6 ether, a base and a solvent:

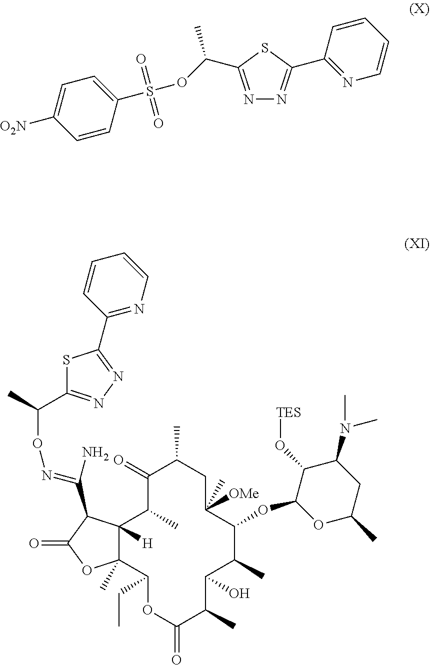

A wide variety of bases and solvents can be used in this step. Typical, non-limiting examples of bases that can be used in this reaction include potassium hydroxide, sodium hydroxide, lithium hydroxide, sodium carbonate, potassium carbonate, cesium carbonate, sodium methoxide, potassium tert-butoxide, sodium ethoxide, pyridine, triethylamine, 4-dimethylaminopyridine, N,N-diisopropylethylamine, or a mixture thereof. Typical, non-limiting examples of solvents that can be used in this step include isopropyl alcohol, methanol, ethanol, dichloromethane, acetone, N,N-dimethylformamide, acetonitrile, N-methylpyrrolidine, tetrahydrofuran, dimethyl sulfoxide, or a mixture thereof. The reaction may be carried out at a wide range of temperatures. In some embodiment, this reaction is carried out at a temperature between 0° C. to 50° C. In some other embodiments, this reaction is carried out at a temperature between 30° C. to 40° C.

Step (i): Preparation of a Compound of Formula (XII)

In general, the compound of Formula (XII) is obtained by reacting the compound of Formula (XI) with N-chlorosuccinimide in presence of dimethyl sulfide, a base and a solvent:

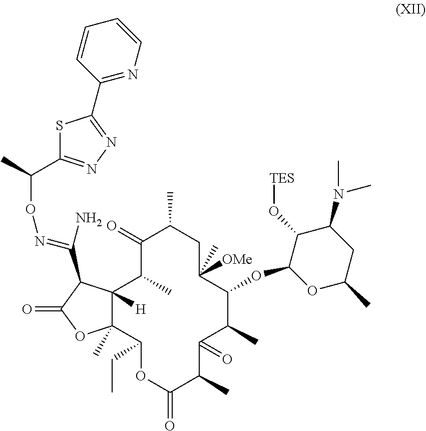

A wide variety of bases and solvents can be used in this step. Typical, non-limiting examples of bases that can be used in this reaction include N,N-diisopropylethylamine, triethylamine, 1,8-diazabicyclo[5.4.0]undec-7-ene, 1,5-diazabicyclo(4.3.0)non-5-ene, or a mixture thereof. Typical, non-limiting examples of solvents that can be used in this step include dichloromethane, toluene, acetonitrile, N,N-dimethylformamide, N-methylpyrrolidine, tetrahydrofuran, dimethyl sulfoxide, or a mixture thereof. The reaction may be carried out at a wide range of temperatures. In some embodiment, this reaction is carried out at a temperature between −20° C. to 30° C. In some other embodiments, this reaction is carried out at a temperature between 0° C. to −10° C.

Step (i): Preparation of a Compound of Formula (I)

In general, the compound of Formula (I) is obtained by de-protecting the compound of Formula (XII) in presence of hydrochloric acid and a solvent. A wide variety of solvents can be used in this step. Typical, non-limiting examples of solvents that can be used in this step include methanol, ethanol, isopropyl alcohol, or a mixture thereof. The reaction may be carried out at a wide range of temperatures. In some embodiment, this reaction is carried out at a temperature between 0° C. to 50° C. In some other embodiments, this reaction is carried out at a temperature between 30° C. to 40° C.

In another general aspect, there is provided a process for preparing a compound of Formula (I),

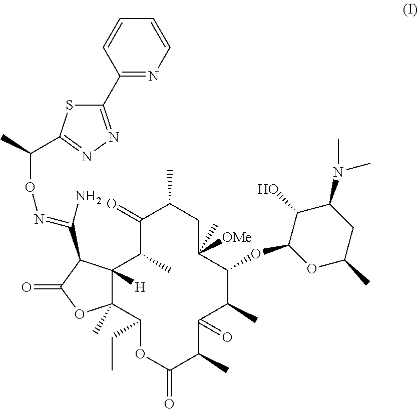 [SMILES: CC[C@@H]1OC(=O)[C@H](C)C(=O)[C@H](C)[C@@H](O[C@@H]2O[C@H](C)C[C@H](N(C)C)[C@H]2O)[C@](C)(OC)C[C@@H](C)C(=O)[C@H](C)[C@H]2[C@H](/C(N)=N/O[C@@H](C)c3nnc(-c4ccccn4)s3)C(=O)O[C@@]21C]

said process comprising:

(a) obtaining a compound of Formula (III) by reacting a compound of Formula (II) with triethylsilyl chloride in presence of triethylamine, 4-dimethylaminopyridine and N,N-dimethylformamide;

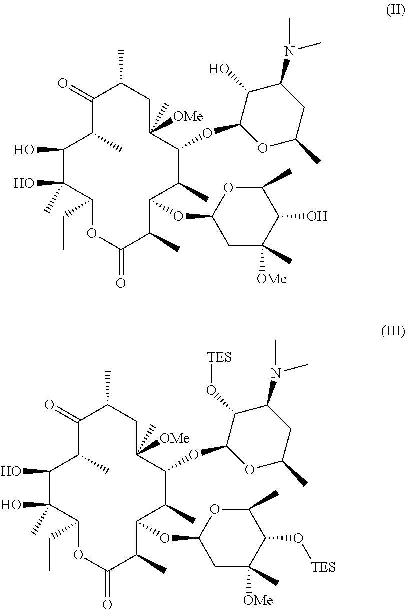

(b) obtaining a compound of Formula (IV) by reacting the compound of Formula (III) with triphosgene in presence of pyridine and dichloromethane;

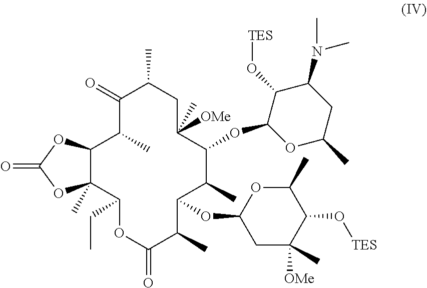

(c) obtaining a compound of Formula (V) by reacting the compound of Formula (IV) with 1,8-diazabicyclo[5.4.0]undec-7-ene in presence of acetone;

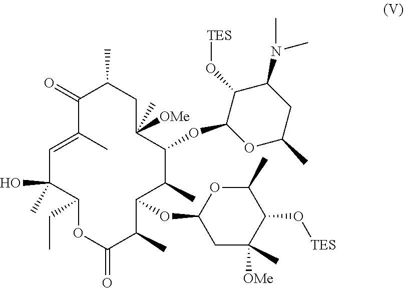

(d) obtaining a compound of Formula (VI) by reacting the compound of Formula (V) with chloroacetic acid and 4-dimethylaminopyridine in presence of N,N′-dicyclohexylcarbodiimide and a dichloromethane;

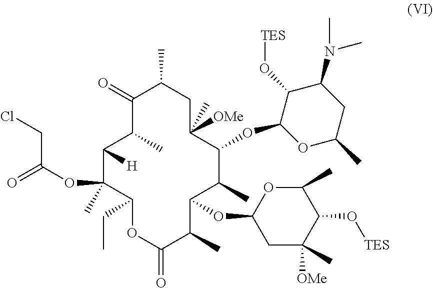

(e) obtaining a compound of Formula (VII) by reacting the compound of Formula (VI) with trimethylsilyl cyanide in presence of cesium carbonate, methanol and N,N-dimethylformamide;

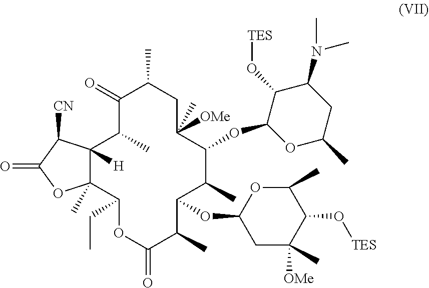

(f) obtaining a compound of Formula (VIII) by treating the compound of Formula (VII) with hydrochloric acid in presence of methanol; followed by treatment with triethysilyl chloride in presence of triethyl amine, 4-dimethylaminopyridine and N,N-dimethylformamide;

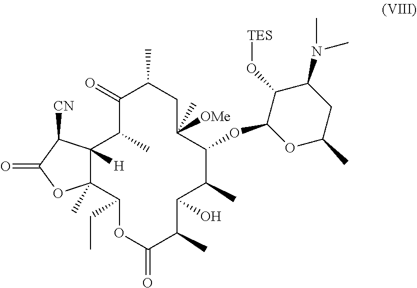

(g) obtaining a compound of Formula (IX) by treating the compound of Formula (VIII) with hydroxylamine hydrochloride in presence of sodium carbonate and methanol;

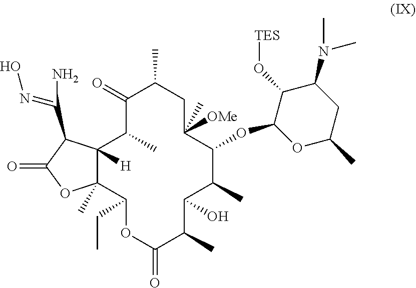

(h) obtaining a compound of Formula (XI) by treating the compound of Formula (IX) with a compound of Formula (X) in presence of 18-crown-6 ether, potassium hydroxide and isopropyl alcohol;

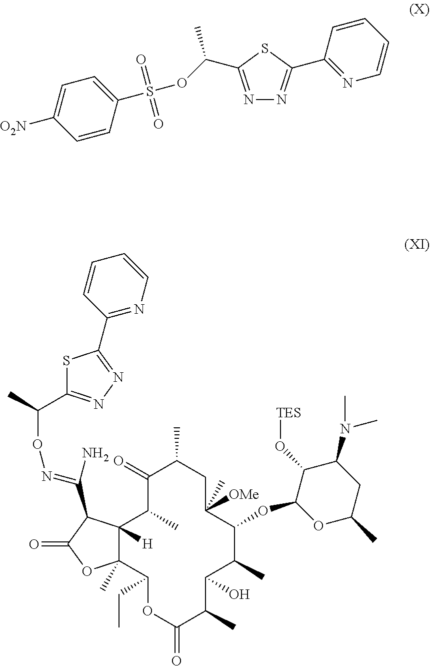

(i) obtaining a compound of Formula (XII) by reacting the compound of Formula (XI) with N-chlorosuccinimide in presence of dimethyl sulfide, N,N-diisopropylethylamine and dichloromethane and toluene; and

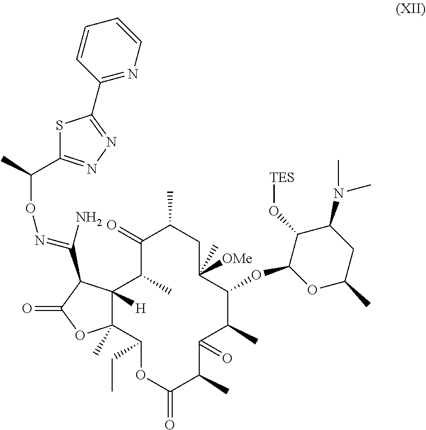

(j) obtaining the compound of Formula (I) by de-protecting the compound of Formula (XII) in presence of methanol and hydrochloric acid.

A wide variety of other reagents which can bring about these functional transformations can be used. It will be readily apparent to one skilled in the art that varying substitutions and modifications may be made to the invention disclosed herein without departing from the scope and spirit of the invention. For example, those skilled in the art will recognize that the invention may be practiced using a variety of different compounds within the described generic descriptions.

### EXAMPLES

The following examples illustrate the embodiments of the invention that are presently best known. However, it is to be understood that the following are only exemplary or illustrative of the application of the principles of the present invention. Numerous modifications and alternative compositions, methods, and systems may be devised by those skilled in the art without departing from the spirit and scope of the present invention. The appended claims are intended to cover such modifications and arrangements. Thus, while the present invention has been described above with particularity, the following examples provide further detail in connection with what are presently deemed to be the most practical and preferred embodiments of the invention.

### Example 1

### Preparation of a Compound of Formula (I)

Step 1: Preparation of a Compound of Formula (III)

A mixture of a compound of Formula (II) (100 gm) in dimethylformamide (300 ml) was cooled to 5-10° C. and to this cooled suspension were added triethylamine (47.5 gm) and 4-dimethylaminopyridine (DMAP, 49 gm). To the resultant reaction mass, triethylsilyl chloride (60 gm) was added in about 60 to 90 minutes under stiffing and the reaction was monitored using HPLC. After completion, the reaction contents were quenched with water (100 ml) and methanol (900 ml) was added and the reaction contents were stiffed for 1 hour. The compound of Formula (III) obtained as solids was washed with water-methanol mixture (1:1, 200 ml), and then dried at about 60° C. for 8 hours. (Yield: 92.3%; HPLC purity: 98%)

Step 2: Preparation of a Compound of Formula (IV)

A solution of a compound of Formula (III) (110 gm) in dichloromethane (550 ml) was cooled to about 5° C. To the cooled solution, was added pyridine (26 gm), followed by addition of a solution of triphosgene (16.7 gm) in dichloromethane (110 ml) under stiffing in about 60 to 120 minutes. The stiffing was continued for 3 hours and reaction was monitored with the help of HPLC. After completion of the reaction, water (660 ml) was added slowly to the reaction mixture to obtain separate organic and aqueous layers. The organic layer was washed with water (660 ml), and then the organic solvent was removed by distillation, and then striped out using Acetone (110) ml. The reaction mass was degassed to get a compound of Formula (IV), which was used as such in further reactions.

Step 3: Preparation of a Compound of Formula (V)

A solution of a compound of Formula (IV) obtained in Step 2 above in acetone (680 ml) was added with 1,8-diazabicyclo[5.4.0]undec-7-ene (DBU, 34 gm) under stirring at about 25 to 30° C. and the reaction mass was refluxed for about 6 to 8 hours. The reaction was monitored with the help of the HPLC. After completion of the reaction, the reaction contents were cooled to about 25-30° C. and water (680 ml) was added to the contents and stirred for 60 minutes. The compound of Formula (V) so obtained was washed with acetone: water mixture (220 ml, 1:1) and dried for about 8-10 hours at about 60° C. (Yield: 98%; HPLC Purity=97%)

Step 4: Preparation of a Compound of Formula (VI)

A solution of a compound of Formula (V) (100 gm) in dichloromethane (500 ml) was added 4-dimethylaminopyridine (6 gm) and the solution was cooled to about 0-10° C. To the cooled solution, was added N,N′-dicyclohexylcarbodiimide (DCC, 43 gm) and a solution of chloroacetic acid (20 gm) in dichloromethane (100 ml) in about 30 to 60 minutes and the entire reaction contents were stirred for about 60 minutes. The reaction was monitored with the help of HPLC. After completion of the reaction, the contents were filtered and the solids were washed with dichloromethane (200). The filtrate was washed with water (2×500 ml). The organic layer was distilled out at about 40° C. under vacuum, and the residue was stripped out using methanol (100 ml). After degassing for about 30 minutes, the compound of Formula (VI) was obtained by addition of methanol (400 ml) and filtered the obtained slurry. The isolated product was washed with methanol (200 ml) and dried under vacuum for about 8 hours at about 60° C. (Yield: 85%; HPLC Purity: 97%)

Step 5: Preparation of a Compound of Formula (VII)

To a mixture of a compound of Formula (VI) (50 gm) in dimethylformamide (250 ml), was added cesium carbonate (18.9 gm), trimethylsilyl cyanide (14.38 gm) and methanol (4.64 gm), and the reaction mass was stirred for about 3 to 4 hours at about 40 to 45° C. The reaction progress was monitored with the help of HPLC. After completion of the reaction, the reaction mass was quenched with water (250 ml) and slurry so obtained was filtered to obtain the compound of Formula (VII), which was further washed with warm water (250 ml) and then dried under vacuum at about 60° C. for about 8 hours (Yield: 97%; HPLC Purity: 97%).

Step 6: Preparation of a Compound of Formula (VIII)

A solution of compound of Formula (VII) (45 gm) in methanol (180 ml) was added aqueous hydrochloric acid (2N, 45 ml) and the contents were stirred at about 35 to 40° C. for about 3 hours. The reaction progress was monitored with the help of HPLC. After completion of the reaction, water (450 ml) and cyclohexane (180 ml) was added the reaction mass. Layers were separated, and aqueous layer was brought to a pH of 8-9 with 10% sodium hydroxide solution. The aqueous layer was extracted with dichloromethane (765 ml). The dichloromethane was distilled out under vacuum, degassed to obtain a semi solid product which was then dissolved in dimethylformamide (75 ml) and the solution was cooled to about 5-10° C. To this cooled solution, triethylamine (8 gm) and 4-dimethylaminopyridine (DMAP, 9.6 gm) were added followed by addition of triethylsilyl chloride (14 gm) over about 60 minutes. The reaction was monitored with the help of HPLC. After completion, the reaction was quenched with methanol (84 ml) and then the entire reaction mass was slowly added to water (530 ml) and stirred for 1 hour at about 25-30° C. followed by cooling to 10-15° C. The slurry so obtained was filtered to obtain the compound of Formula (VIII) in crude form, which was washed with a mixture of water and methanol (1:1, 34 ml) and then dried at about 60° C. for about 8 hours (Yield: 95%; HPLC purity: 85%).

The crude compound of Formula (VIII) was by suspending in cyclohexane (135 ml) under stiffing for about 1 hour at a temperature of about 25-30° C., followed by cooling to about 10-15° C. and stiffing at that temperature for another for 1 hour, then filtered the slurry to obtain solid which were washed with cyclohexane (34 ml), to obtain the pure compound of Formula (VIII). The product was dried at about 60° C. for 4 hours (Yield: 85%; HPLC purity: 95%)

Step-7: Preparation of a Compound of Formula (IX)

To a stirred suspension of a compound of Formula (VIII) (20 gm) in methanol (160 ml), sodium bicarbonate (6.76 gm), and hydroxylamine hydrochloride (5.6 gm) were added and the contents were stirred at about 35-40° C. for about 8 hours. The reaction progress was monitored with the help of HPLC. After completion, activated charcoal was added to the contents and stirred for 30 minutes, then filtered on celite bed to remove activated charcoal, washed the bed with methanol (40 ml) and collected the filtrate. To this filtrate, water (200 ml) was added in about 30-60 minutes at a temperature of about 25-30° C., and the slurry was filtered to obtain solid. The solid were washed with water-methanol mixture (1:1, 40 ml). The resulting compound of Formula (IX) was dried at about 60° C. for 8 hours under vacuum (Yield: 95%; HPLC purity: 98%)

Step-8: Preparation of a Compound of Formula (XI)

To the mixture of a compound of Formula (IX) (20 gm) in isopropyl alcohol (100 ml) was added 18-crown-6 ether (1.34 gm), potassium hydroxide powder (1.85 gm) and compound of Formula (X) (10 gm). The reaction mass was stirred at about 35-40° C. for 30 minutes and the reaction progress was monitored with the help of HPLC. After completion of the reaction, reaction mixture was cooled to room temperature, diluted with dichloromethane (200 ml) and water (200 ml). This mixture was stirred for 15 minutes and layers were separated. The organic solvent was removed by distillation, and then striped out using methanol (120 ml). The reaction mixture was degassed to get a semi solid product and then added methanol (100 ml). The contents were warmed to about 45-50° C. To this solution water (20 ml) was added slowly to get precipitation and stirred for 15 minutes. Reaction mixture was cooled to about 10 to 15° C., stirred for 1 hour and filtered. The compound of Formula (XI) so obtained was washed with a methanol:water mixture (40 ml, 1:1) and dried for another about 8 hours at about 60° C. (Yield: 85%; HPLC purity: 95%)

Step-9: Preparation of a Compound of Formula (XII)

A mixture of N-chlorosuccinimide (6.5 gm) in dichloromethane (57 ml) and toluene (76 ml) was cooled to about 0 to −10° C. To this cooled suspension was added a solution of dimethyl sulfide (3.3 gm) in toluene (10 ml) maintaining the temperature to about −5 to −10° C. The suspension was stirred for about 30 minutes, and then a solution of a compound of Formula (XI) (19 gm) in toluene (122 ml). The resulting reaction mass was stirred for about 90 minutes, and then N,N-diisopropylethylamine (6.5 gm) was added and the stirring was continued for another 30 minutes. The reaction progress was monitored with the help of HPLC. After completion of the reaction, water (190 ml) was added to the reaction mass and layers were separated. The organic layer containing product was washed with aqueous 4% sodium bicarbonate solution (190 ml) and then with water (190 ml). The organic solvent was removed under vacuum and stripped out using methanol (120 ml), and then degassed. The compound of Formula (XII) so obtained was used as such in the further reaction.

Step-10: Preparation of a Compound of Formula (I)

To a solution of a compound of Formula (XII) in methanol (57 ml) was added 2N hydrochloric acid (38 ml). The reaction mass was stirred for 3 hours at about 35-40° C. and the reaction was monitored by HPLC. After completion of the reaction, water (38 ml) was added to the reaction mass and the contents were extracted using toluene (114 ml). To the aqueous layer was added to methanol (38 ml) along with activated charcoal (1 gm) and the contents were stifled for 15 minutes, filtered through celite bed and the filtrate was collected in a flask. The pH of this filtrate was adjusted to about 8.5 to 9.0 using 10% sodium hydroxide solution, and the contents were stirred for 1 hour at about 25-30° C. The crude compound of Formula (I) so obtained was filtered, suspended in methanol (95 ml) at about 50° C. under stirring for about 15 minutes, then cooled to about 25-30° C. and stifled for another 1 hour, washed with methanol (40 ml) and dried under vacuum at about 60° C. for about 10 hours to obtain a pure compound of Formula (I) (Yield: 65%; Specific rotation [α]₂₅D (c 0.5, acetonitrile): −60.14°).

1. A process for preparing a compound of Formula (I), 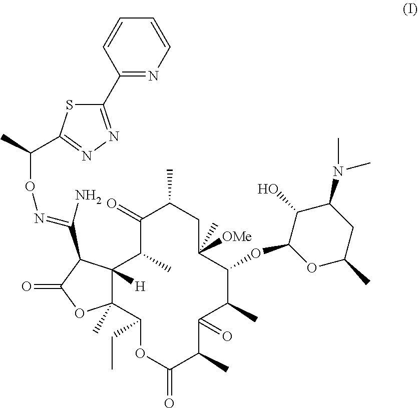 [SMILES: CC[C@@H]1OC(=O)[C@H](C)C(=O)[C@H](C)[C@@H](O[C@@H]2O[C@H](C)C[C@H](N(C)C)[C@H]2O)[C@](C)(OC)C[C@@H](C)C(=O)[C@H](C)[C@H]2[C@H](/C(N)=N/O[C@@H](C)c3nnc(-c4ccccn4)s3)C(=O)O[C@@]21C]
said process comprising:
(a) obtaining a compound of Formula (III) by reacting a compound of Formula (II) with triethylsilyl chloride in presence of a base and a solvent; 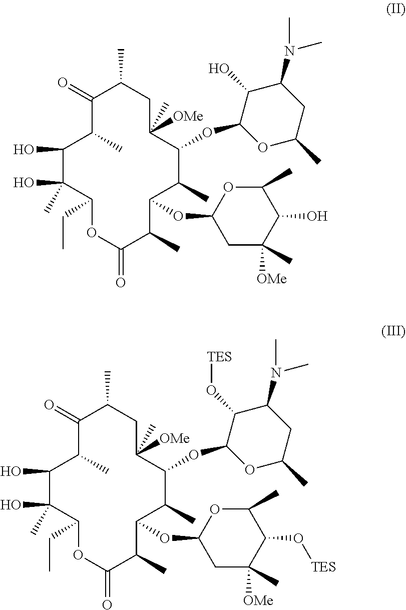
(b) obtaining a compound of Formula (IV) by reacting the compound of Formula (III) with triphosgene in presence of a base and a solvent; 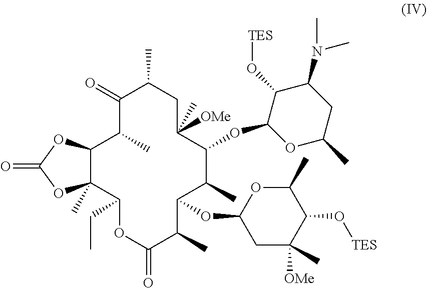
(c) obtaining a compound of Formula (V) by reacting the compound of Formula (IV) with a base in presence of a solvent; 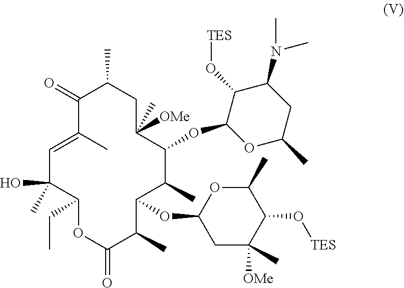
(d) obtaining a compound of Formula (VI) by reacting the compound of Formula (V) with chloroacetic acid and 4-dimethylaminopyridine in presence of a coupling agent and a solvent; 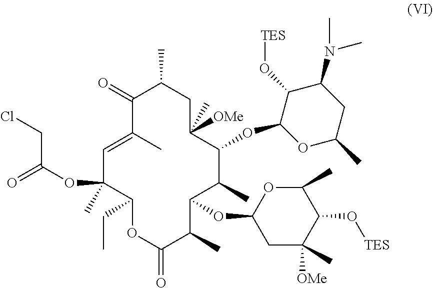
(e) obtaining a compound of Formula (VII) by reacting the compound of Formula (VI) with trimethylsilyl cyanide in presence of a base, an activating agent and a solvent; 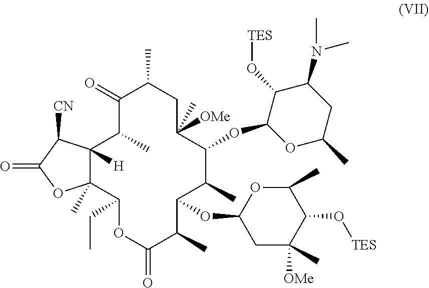
(f) obtaining a compound of Formula (VIII) by treating the compound of Formula (VII) with hydrochloric acid in presence of a solvent; followed by treatment with triethysilyl chloride in presence of a base and a solvent; 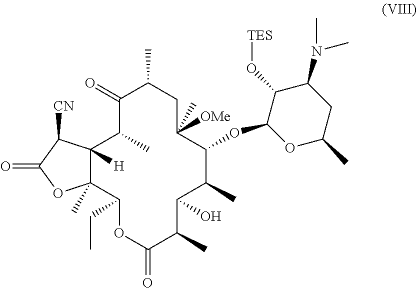
(g) obtaining a compound of Formula (IX) by treating the compound of Formula (VIII) with hydroxylamine hydrochloride in presence of a base and a solvent; 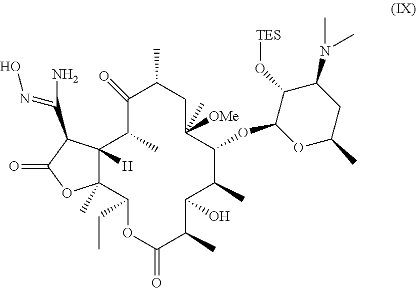
(h) obtaining a compound of Formula (XI) by treating the compound of Formula (IX) with a compound of Formula (X) in presence of 18-crown-6 ether, a base and a solvent; 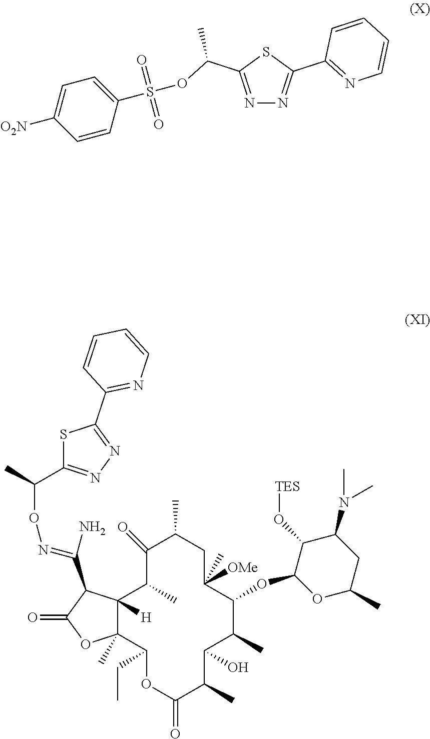
(i) obtaining a compound of Formula (XII) by reacting the compound of Formula (XI) with N-chlorosuccinimide in presence of dimethyl sulfide, a base and a solvent; and 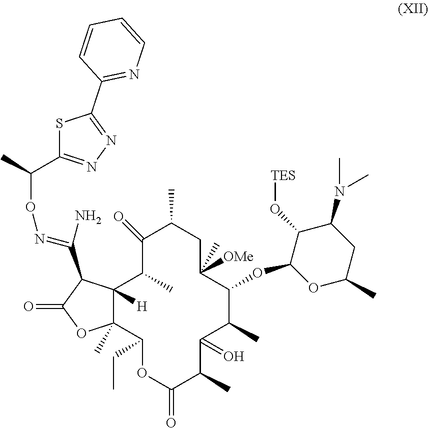
(j) obtaining the compound of Formula (I) by de-protecting the compound of Formula (XII).

2. The process according to claim 1, wherein the base used in step (e) is selected from cesium carbonate, sodium carbonate, potassium carbonate, sodium hydroxide, potassium hydroxide, lithium hydroxide, sodium methoxide, potassium tert-butoxide, sodium ethoxide, or a mixture thereof.

3. The process according to claim 1, wherein the solvent used in step (e) is selected from a N,N-dimethylformamide, N-methylpyrrolidine, tetrahydrofuran, ethylacetate, acetone, acetonitrile, dimethyl sulfoxide or a mixture thereof.

4. The process according to claim 1, wherein an activating agent used in step (e) is selected from a C₁-C₆ alcohol, water or a mixture thereof.

5. The process according to claim 1, wherein the activating agent used in step (e) is methanol, water or a mixture thereof.

6. A process for preparing a compound of Formula (I), 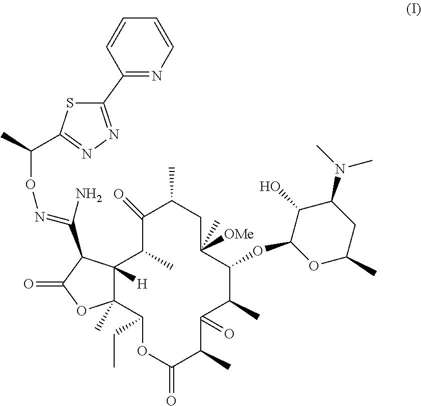 [SMILES: CC[C@@H]1OC(=O)[C@H](C)C(=O)[C@H](C)[C@@H](O[C@@H]2O[C@H](C)C[C@H](N(C)C)[C@H]2O)[C@](C)(OC)C[C@@H](C)C(=O)[C@H](C)[C@H]2[C@H](/C(N)=N/O[C@@H](C)c3nnc(-c4ccccn4)s3)C(=O)O[C@@]21C]
said process comprising:
(a) obtaining a compound of Formula (III) by reacting a compound of Formula (II) with triethylsilyl chloride in presence of triethylamine, 4-dimethylaminopyridine and N,N-dimethylformamide; 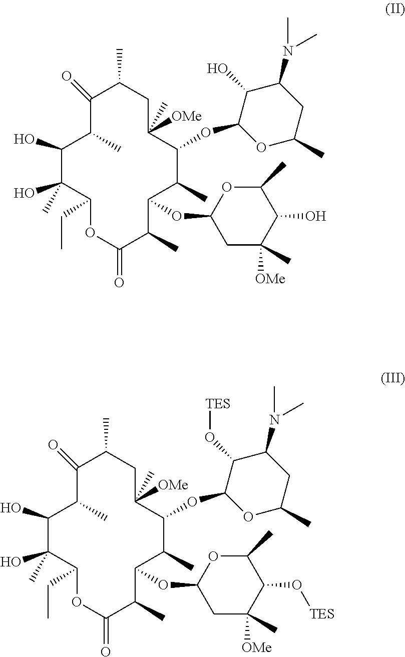
(b) obtaining a compound of Formula (IV) by reacting the compound of Formula (III) with triphosgene in presence of pyridine and dichloromethane; 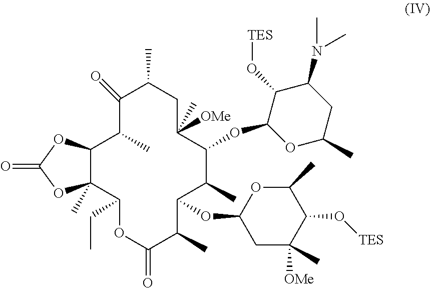
(c) obtaining a compound of Formula (V) by reacting the compound of Formula (IV) with 1,8-diazabicyclo[5.4.0]undec-7-ene in presence of acetone; 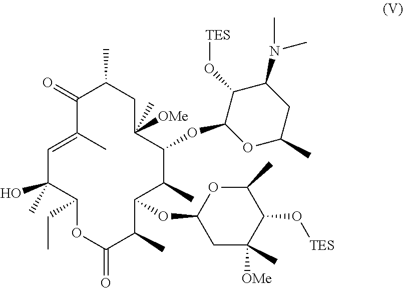
(d) obtaining a compound of Formula (VI) by reacting the compound of Formula (V) with chloroacetic acid and 4-dimethylaminopyridine in presence of N,N′-dicyclohexylcarbodiimide and a dichloromethane; 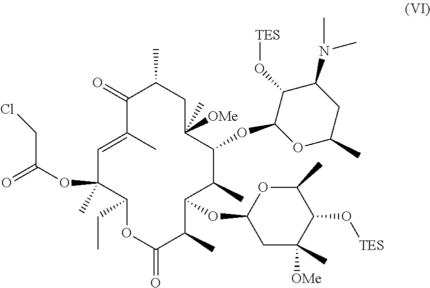
(e) obtaining a compound of Formula (VII) by reacting the compound of Formula (VI) with trimethylsilyl cyanide in presence of cesium carbonate, methanol and N,N-dimethylformamide; 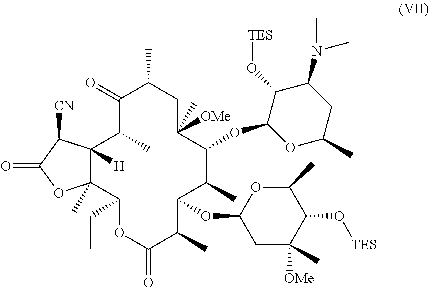
(f) obtaining a compound of Formula (VIII) by treating the compound of Formula (VII) with hydrochloric acid in presence of methanol; followed by treatment with triethysilyl chloride in presence of triethyl amine, 4-dimethylaminopyridine and N,N-dimethylformamide; 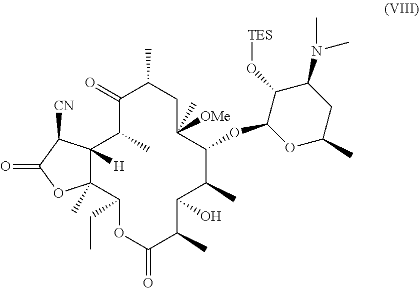
(g) obtaining a compound of Formula (IX) by treating the compound of Formula (VIII) with hydroxylamine hydrochloride in presence of sodium carbonate and methanol; 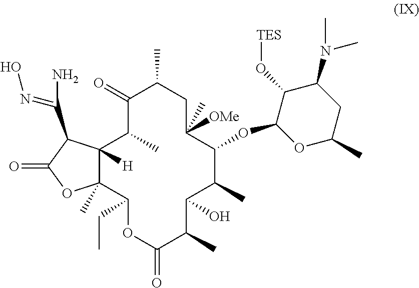
(h) obtaining a compound of Formula (XI) by treating the compound of Formula (IX) with a compound of Formula (X) in presence of 18-crown-6 ether, potassium hydroxide and isopropyl alcohol; 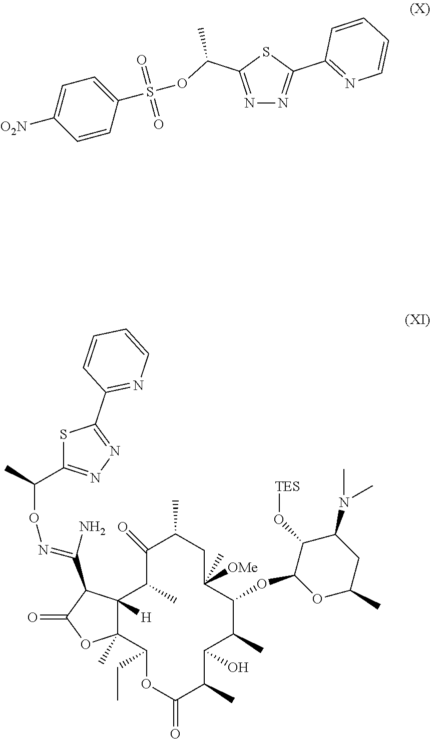
(i) obtaining a compound of Formula (XII) by reacting the compound of Formula (XI) with N-chlorosuccinimide in presence of dimethyl sulfide, N,N-diisopropylethylamine and dichloromethane and toluene; and 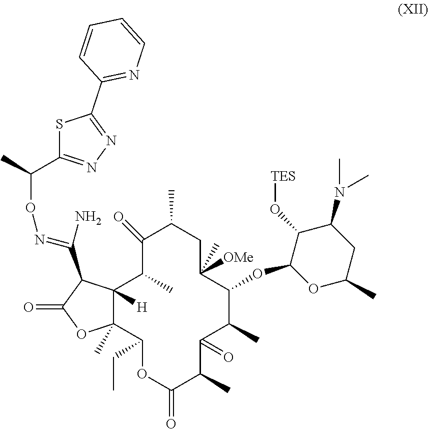
(j) obtaining the compound of Formula (I) by de-protecting the compound of Formula (XII) in presence of methanol and hydrochloric acid.

7. A process for preparing a compound of Formula (VII), said process comprising reacting a compound of Formula (VI) with trimethylsilyl cyanide in presence of a base, an activating agent and a solvent.

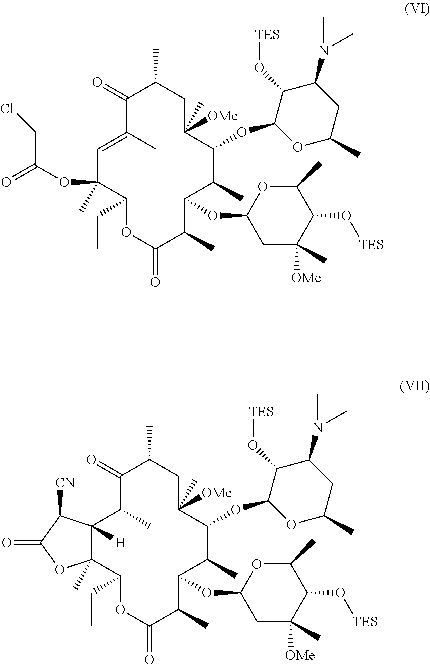

8. The process according to claim 7, wherein the base is selected from cesium carbonate, sodium carbonate, potassium carbonate, sodium hydroxide, potassium hydroxide, lithium hydroxide, sodium methoxide, potassium tert-butoxide, sodium ethoxide, or a mixture thereof.

9. The process according to claim 7, wherein an activating agent is selected from a C₁-C₆ alcohol, water or a mixture thereof.

10. The process according to claim 7, wherein an activating agent is methanol.

11. The process for preparing the compound of Formula (VII), said process comprising reacting a compound of Formula (VI) with trimethylsilyl cyanide in presence of cesium carbonate, methanol and N,N-dimethylformamide. [chemical structure]
